(19) United States
(12) Patent Application Publication
(10) Pub. No.: US 2022/0153684 A1
(43) Pub. Date: May 19, 2022

(54) PROCESS FOR PREPARATION OF FINGOLIMOD HYDROCHLORIDE

(71) Applicant: SHIVALIK RASAYAN LIMITED, Bhiwadi (IN)

(72) Inventors: AKSHAY KANT CHATURVEDI, Bhiwadi (IN); SATYENDRA SINGH, Bhiwadi (IN); SATBIR SINGH, Bhiwadi (IN); GAJENDRA KUMAWAT, Bhiwadi (IN)

(73) Assignee: SHIVALIK RASAYAN LIMITED, Bhiwadi (IN)

(21) Appl. No.: 17/513,939

(22) Filed: Oct. 29, 2021

(30) Foreign Application Priority Data
Nov. 13, 2020 (IN) 202011049584

(51) Int. Cl. C07C 213/08; C07C 219/22

(52) U.S. Cl. CPC C07C 213/08; C07C 219/22

(57) ABSTRACT

The present invention relates to a process for the preparation of the active pharmaceutical ingredient Fingolimod Hydrochloride (I) and its highly pure intermediate [2-acetamido-2-(acetyloxy methyl)-4-phenylbutyl] acetate(II)

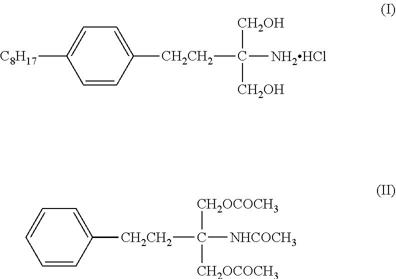

### FIELD OF THE INVENTION

The present invention relates to a process for the preparation of the active pharmaceutical ingredient Fingolimod Hydrochloride (I) and its highly pure intermediate [2-acetamido-2-(acetyloxy methyl)-4-phenylbutyl] acetate(II)

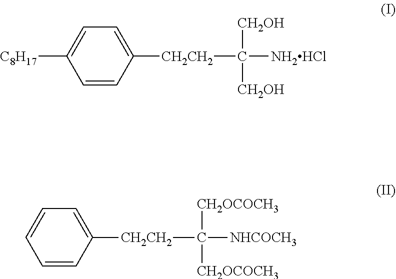

The present invention also relates to the highly pure intermediate [2-acetamido-2-(acetyloxy methyl)-4-phenylbutyl] acetate(II) having purity exceeding 98% (by HPLC).

### BACKGROUND OF THE INVENTION

Fingolimod hydrochloride (FTY720) has the IUPAC name as 2-amino-2-[2-(4-octylphenyl) ethyl] propane-1,3-diol hydrochloride and has the following structure:

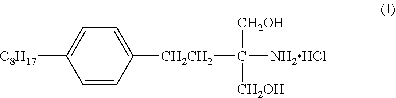

Fingolimod is a sphingosine 1-phosphate receptor modulator indicated and approved for the treatment of relapsing-remitting multiple sclerosis. Fingolimod hydrochloride capsule with proprietary name ‘GILENYA’ and strength of 0.50 mg was approved by USFDA on Sep. 21, 2010 for oral administration.

Fujita et al. U.S. Pat. No. 5,604,229 is the first disclosure of the Fingolimod, its processes and other related compounds. Patent discloses 2-Amino-1, 3-propanediol compounds of the formula

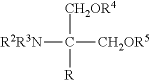

wherein R is an optionally substituted straight or branched carbon chain, an optionally substituted aryl, an optionally substituted cycloalkyl or the like, and R2, R3, R4 and R5 are the same or different and each is a hydrogen, an alkyl, an aralkyl, an acyl or an alkoxycarbonyl, pharmaceutically acceptable salts thereof and immune suppressants comprising these compounds as active ingredients. The 2-amino-1, 3-propanediol compounds disclosed immunosuppressive action and are useful for suppressing rejection in organ or bone marrow transplantation, prevention and treatment of autoimmune diseases or as reagents for use in medicinal and pharmaceutical fields.

Kunitomo Adachi et al. in U.S. Pat. No. 6,214,873B1 discloses the preparation of 2-acetamido-1, 3-diacetoxy-2-(2-phenylethyl) propane also known as [2-acetamido-2-(acetyloxy methyl)-4-phenylbutyl] acetate(II) from diethyl acetamidomalonate reacting with 2-phenylethyl bromide in presence of sodium hydride to give diethyl 2-acetamido-2-(2-phenylethyl) malonate.

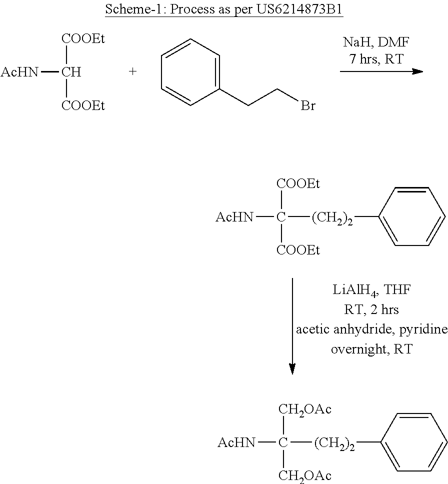

On further hydrolysis with lithium aluminum hydride and on acetylation results in the desired compound. The process involves the use of sodium hydride and lithium aluminium hydride which are not easy to handle at industrial scale resulting in the process to be unamenable to commercial scale.

Adachi Kunitomo et al. in JP 4079505 B2 discloses the process of preparation of 2-amino-2-(2-(4-octylphenyl)ethyl)propane-1,3-diol from diethyl acetylamide malonate is reacted with (2-haloethyl) benzene in the presence of a sodium hydride to obtain diethyl 2-acetylamide-2-phenylethyl malonate.

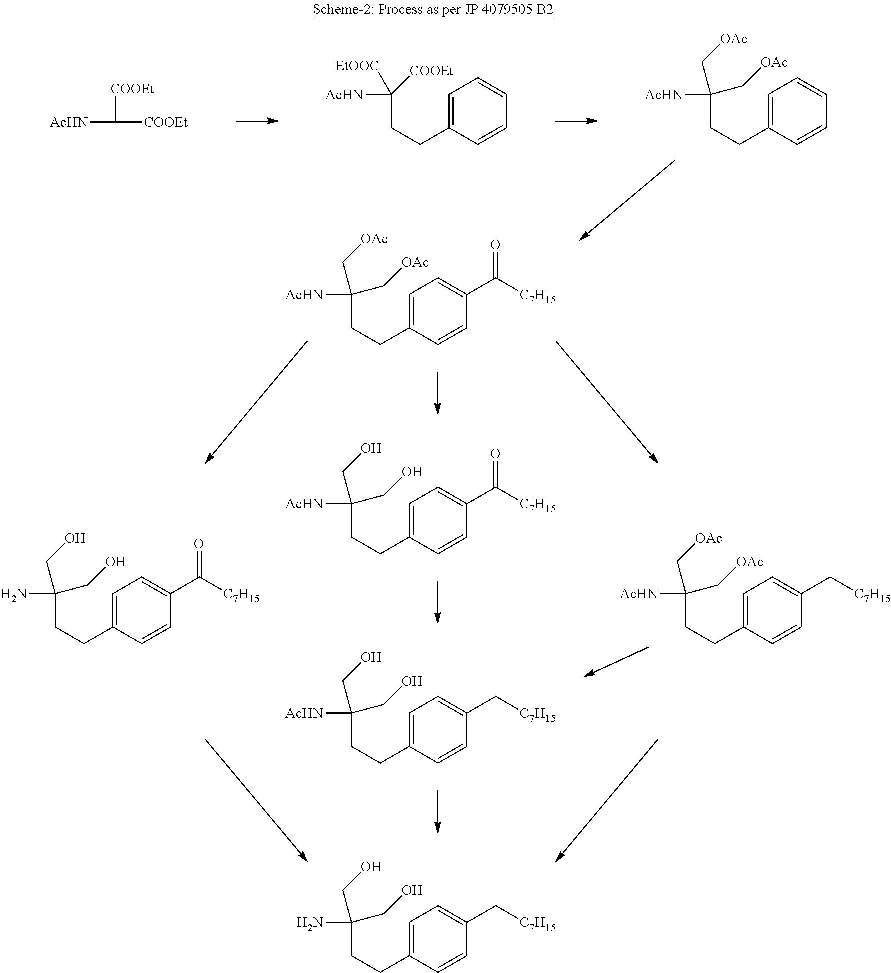

After reducing this with lithium aluminium hydride, further acetylated to 2-acetylamide-2-acetoxymethyl-4-phenylbutyl acetate, and then an octanoyl group is introduced. The process involves several number of steps resulting in poor yield.

Chen Xiaoxiang et al. in WO2012031466A1 discloses the process for the preparation of 2-(p-octylphenylethyl)-2-amino propanediol derivatives involving the preparation of 2-acetylamino 2-(2-phenylethyl)-1,3-propanediol diacetate from acetamido malonic acid diethyl ester by reacting with phenylethyliodide in presence of sodium ethoxide, reducing with lithium aluminium hydride and further acetylating.

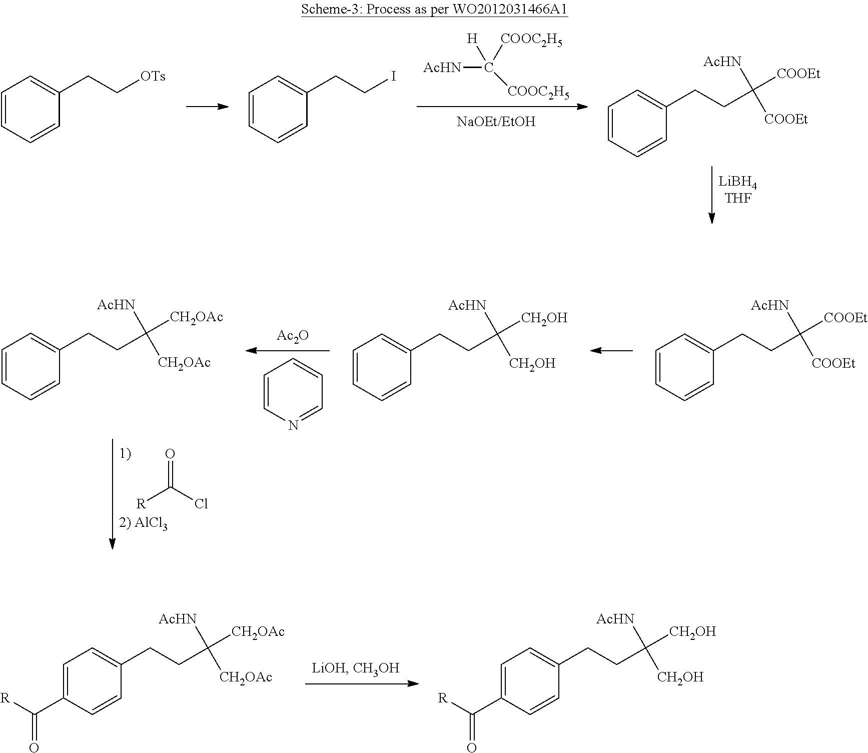

The process involves sodium ethoxide which is to be used when freshly prepared and the overall yield was observed very low with low purity levels.

Chen Xiaoxiang et al. in CN102887834 A discloses the process of preparation of 1-[4-[3-amino-4-hydroxy-3-(hydroxymethyl)butyl]phenyl]-1-acetoxime involving the preparation of 2-acetamido-1, 3-diacetoxy-2-(2-phenylethyl) propane starting from acetamido diethyl malonate reacting with sodium hydride in presence of dimethylformamide which on further reduction with sodium borohydride in presence of ethanol. Reacting N-[1,1-bis (hydroxymethyl)-3-phenylpropyl] acetamide with acetic anhydride in tetrahydrofuran, triethylamine and DMAP.

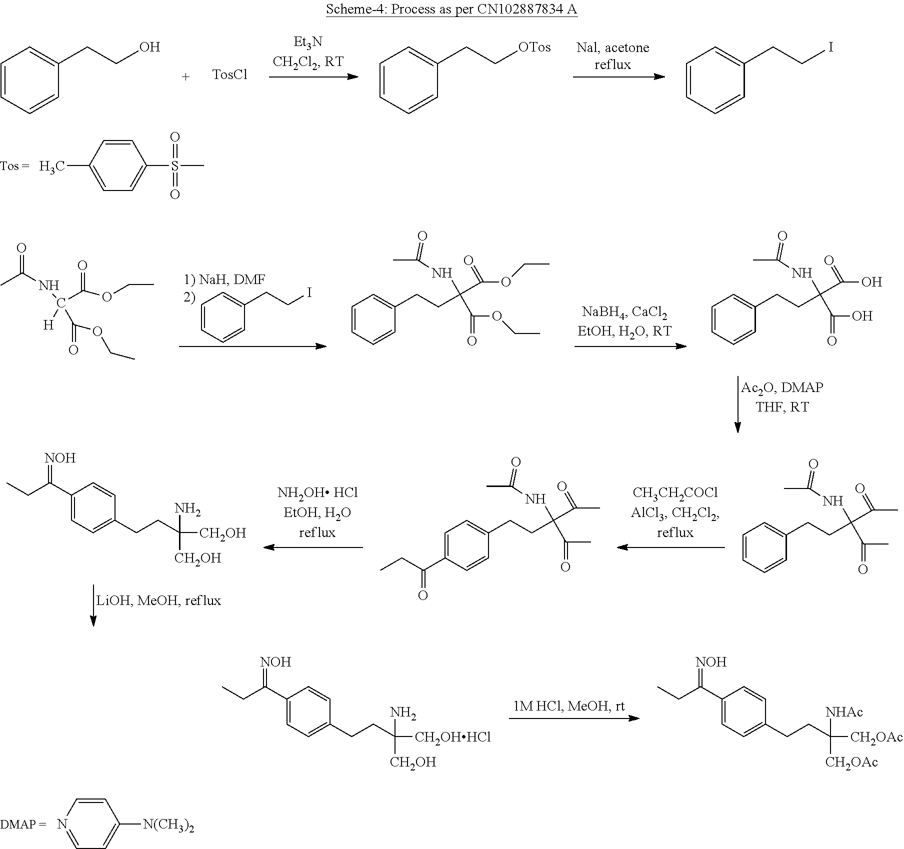

The process is for the preparation of impurities relates to Fingolimod Hydrochloride. The steps are observed to be lengthy and have use of hazardous reagents which are not usable for bulk production.

Shrawat et al. in U.S. Pat. No. 9,732,030B2 discloses the process of preparation of fingolimod and its salts involving the process of preparation of 2-acetamido-2-phenethylpropane-1,3-diyl diacetate starting from the reaction of diethyl acetamido malonate with phenylethylbromide in presence of sodium metal in ethanol to yield diethyl-2-acetamido-2(2-phenyl ethyl) malonate on reducing with lithium aluminium hydride and acetylating with acetic anhydride. More particularly in the step 1 of example 1, on reproducing entity resulted in a desired regioisomer ratio of not exceeding 55-70% (by HPLC), whereby the residue obtained was subjecting to purify by column chromatography over silica gel (230-400 mesh) using an eluent system of ethyl acetate and hexane. The column purified material was still having purity of not exceeding of 72-80%. Said material was used for the step-2 reduction whereby the step-2 product obtained had purity of about 90% (by HPLC).

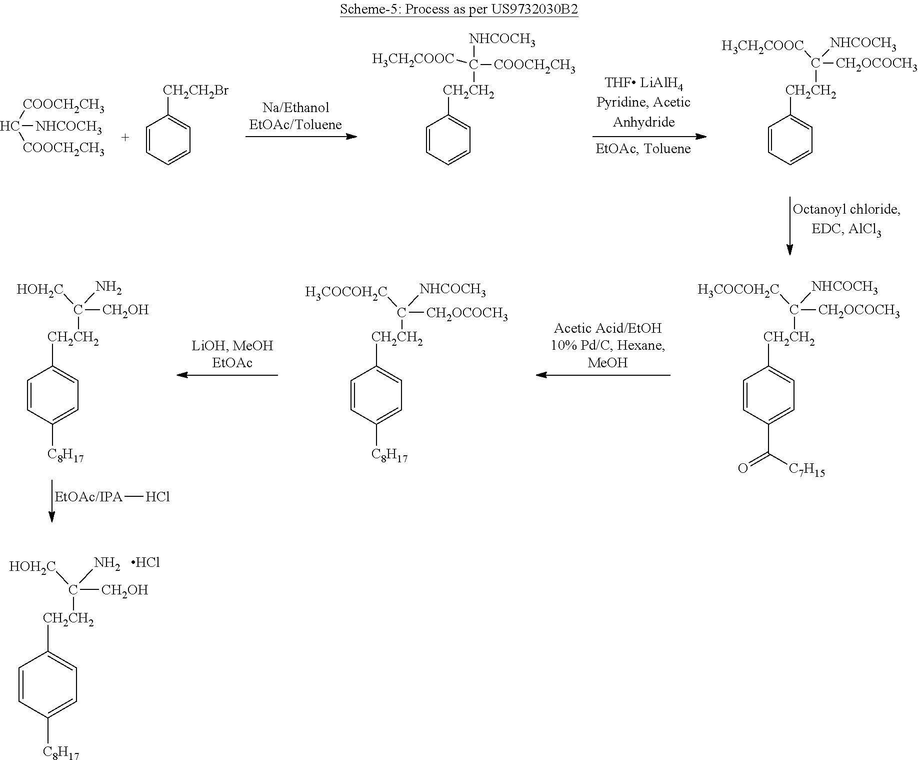

This material was 2 times purified by dissolving in methanol and recrystallized by low temperature to get a purity of >99.5%. The disadvantage of the process is that it was observed the process is very tedious, cumbersome and time consuming method and has several repeated purifications.

Further, in view of the existence of various literature/information known for processes related to preparation of Fingolimod hydrochloride, there exists a need of process/es, which are not only industrially and economically feasible process but also amenable to scale up and provide improved yields & quality.

Within the large number of methods of preparation of Fingolimod Hydrochloride and its intermediates, inventors of the present invention have found the methods, which involve the use of highly pure intermediate [2-acetamido-2-(acetyloxy methyl)-4-phenylbutyl] acetate(II) is highly efficient and industrially feasible.

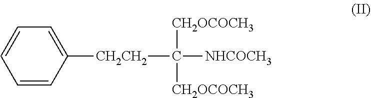

Thus, the inventors of the present application provide a simple and industrially viable process for the preparation of Fingolimod or its hydrochloride involving the use of highly pure intermediate [2-acetamido-2-(acetyloxy methyl)-4-phenylbutyl] acetate (II) by a novel process which is highly efficient and easily up scalable.

### SUMMARY OF THE INVENTION

Particular aspects of the present invention relate to the process for the preparation of the Fingolimod Hydrochloride (I) and its highly pure key intermediate [2-acetamido-2-(acetyloxymethyl)-4-phenylbutyl] acetate(II).

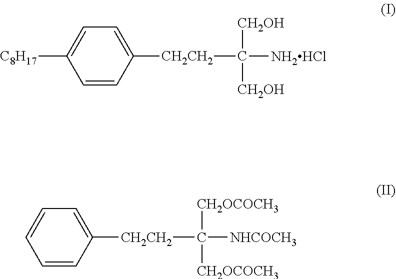

comprising the steps of:

a) reacting diethylacetamidomalonate(VI) with phenylethylhalide(V) in presence of a phase transfer catalyst, base and polar aprotic solvent at temperature ranging between 80-100° C. for a time ranging between 4-8 hours to give diethyl 2-acetamido-2-phenethylmalonate (IV).

b) reacting diethyl 2-acetamido-2-phenethylmalonate(IV) with aqueous NaBH4 at temperature ranging between 30-70° C. to give N-(1-hydroxy-2-(hydroxymethyl)-4-phenylbutan-2-yl) acetamide (III).

c) acetylation of N-(1-hydroxy-2-(hydroxymethyl)-4-phenylbutan-2-yl) acetamide(III) with an acetylating agent in presence of an organic solvent for time duration ranging between 2-6 hrs to give [2-acetamido-2-(acetyloxy methyl)-4-phenylbutyl] acetate (II).

d) the converting [2-acetamido-2-(acetyloxy methyl)-4-phenylbutyl] acetate(II) obtained in step c) to get Fingolimod Hydrochloride of formula (I).

In yet another aspect the present invention also relates to a highly pure key intermediate [2-acetamido-2-(acetyloxy methyl)-4-phenylbutyl] acetate (II) having purity exceeding 98% (by HPLC).

The HPLC method used as per the present invention was performed at column Intersil ODS-3V (250×4.6) 5 μm or equivalent with solvent system of 0.1% trifluoroacetic acid in acetonitrile at a wavelength of 220 nm.

Further particular aspects of the invention are detailed in the description part of the specification, wherever appropriately desired.

### BRIEF DESCRIPTION OF THE DRAWINGS

FIG. 1 is an illustration of HPLC graph of compound of formula (II).

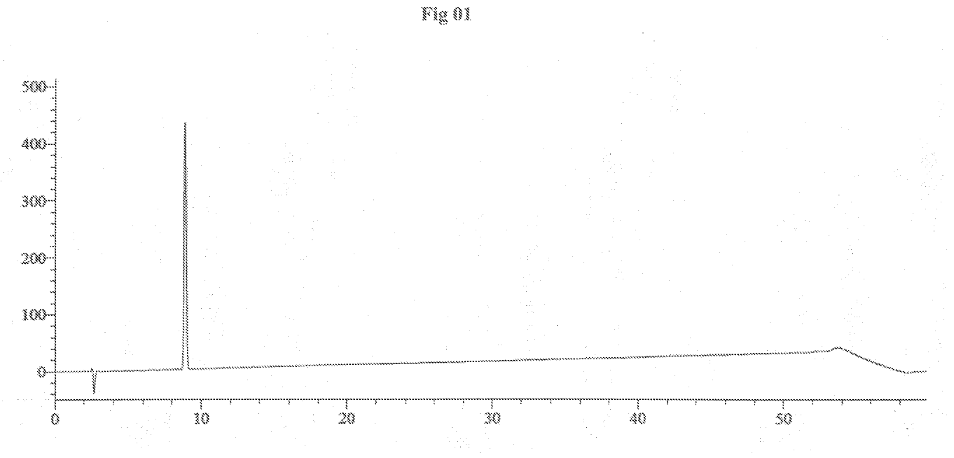

FIG. 2 is an illustration of HPLC graph of compound of formula (I).

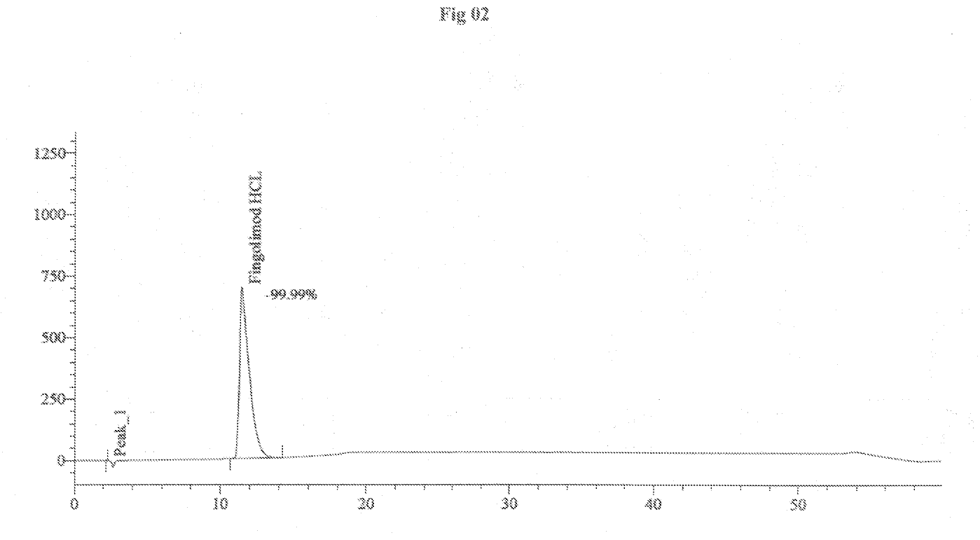

### DETAILED DESCRIPTION

Embodiments according to present invention provides a commercially amenable process for preparing Fingolimod Hydrochloride, which is stable and suitable for preparing therapeutic dosage forms and its highly pure key intermediate [2-acetamido-2-(acetyloxy methyl)-4-phenylbutyl] acetate(II).

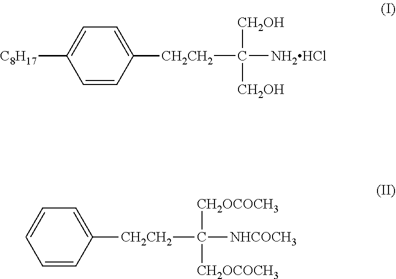

In one embodiment of the present invention, it provides a process for preparing Fingolimod Hydrochloride (I) comprising of the following steps:

a) reacting diethylacetamidomalonate(VI) with phenylethylhalide(V) in presence of a phase transfer catalyst, base and polar aprotic solvent at temperature ranging between 80-100° C. for a time ranging between 4-8 hours to give diethyl 2-acetamido-2-phenethylmalonate (IV).

b) reacting diethyl 2-acetamido-2-phenethylmalonate(IV) with aqueous NaBH4 at temperature ranging between 30-70° C. to give N-(1-hydroxy-2-(hydroxymethyl)-4-phenylbutan-2-yl) acetamide (III).

c) acetylation of N-(1-hydroxy-2-(hydroxymethyl)-4-phenylbutan-2-yl) acetamide(III) with an acetylating agent in presence of an organic solvent for time duration ranging between 2-6 hrs to give [2-acetamido-2-(acetyloxy methyl)-4-phenylbutyl] acetate (II).

d) the converting [2-acetamido-2-(acetyloxy methyl)-4-phenylbutyl] acetate(II) obtained in step c) to get Fingolimod Hydrochloride of formula (I).

Individual steps of the process according to the present invention are detailed herein below.

In step a), the reaction is performed under nitrogen. Diethylacetamidomalonate(VI) is dissolved in a polar aprotic solvent i.e. dimethyl sulfoxide at 25-30° C. followed by addition of base selected from cesium carbonate or lithium carbonate and the phase transfer catalyst for the initiation of the reaction.

The role of base in the condensation reaction was found to be significantly important.

Inventors found that cesium carbonate works as chemo selective inorganic base catalyst. It was observed that particularly cesium carbonate and lithium carbonate as selective catalyst for this step while others alkali carbonates like sodium carbonate and potassium carbonate were found to be ineffective in performing the reaction.

The phase transfer catalyst used in the present invention is tetrabutylammonium bromide followed by the addition of phenylethylhalide(V) under nitrogen atmosphere at 80-100° C. specifically at 80-85° C. to give diethyl 2-acetamido-2-phenethylmalonate (IV).

The phenylethylhalide used in step a) is selected from phenylethylbromide, phenylethyliodide, phenylethylchloride.

In a particular embodiment, it was phenylethylbromide was used.

The molar ratio of phenylethylbromide used was 1=1.05 about molar ratio with respect to of diethylacetamidomalonate.

The use of phase transfer catalyst in this step was found to provide reaction a smoother course resulting in an improved yield.

Inventors observed that a reaction without phase transfer catalyst resulted in about 20-25% lesser yield. The improve yield by using tetrabutylammonium bromide as a phase transfer catalyst appears to be owing to by phasic reaction, wherein both organic and inorganic phase remains in continuous connect during the course of reaction, which appears to be missing in the case of reactions performed without phase transfer catalyst.

In step b) of the present invention, diethyl 2-acetamido-2-phenethylmalonate(IV) is treated with aqueous NaBH₄ which is found to be more safe as compared the lithium aluminium hydride used in the prior art.

In the prior art, it is mentioned that reaction may not proceed with sodium borohydride or may result in low purity and yield as compared to the other reducing agents such as sodium hydride, lithium aluminium hydride, lithium borohydride.

The inventors of the present invention observed that the addition of solid sodium borohydride or in anhydrous environment does not move the reaction to completion however, inventors surprisingly observed that the aqueous solution of sodium borohydride results in completion of reaction with low impurity profile. The use of aqueous solution of sodium borohydride is found to be easy to use in scalable processes. The percentage aqueous solution of sodium borohydride was observed suitable as about 30-70% w/w in aqueous medium.

The step b) of the present invention is performed in aqueous alcoholic medium comprising a ratio of alcohol (C1-C3):water mixture is ranging between 1(diethyl 2-acetamido-2-phenethyl malonate(IV)):10-15 (alcohol) (w/v) and 1(diethyl 2-acetamido-2-phenethylmalonate(IV)):2-3(water) (w/v).

The alcohol which is used in the aqueous alcoholic medium of step b) is selected from methanol, ethanol, n-butanol and isopropanol.

In particular embodiment, it was methanol used in the aqueous alcoholic medium wherein the ratio of methanol:water mixture is ranging between 1(diethyl 2-acetamido-2-phenethyl malonate(IV)):10(methanol) (w/v) and 1(diethyl 2-acetamido-2-phenethylmalonate(IV)): 2(water) (w/v).

The inventors have found that the solvent medium needs to be alcoholic for the reaction to occur in the manner to give desired product. The inventors have investigated methanol, ethanol, 2-propanol, tetrahydrofuran, pyridine for the step b) of the present invention.

Reaction in methanol with aqueous solution of sodium borohydride was observed readily proceed at 30-70° C.

In a particular embodiment, Inventors observed that the reaction proceeds to maximum at 40-60° C. temperature range.

In reaction step c) of the present invention, it is preferred under nitrogen the crude N-(1-hydroxy-2-(hydroxymethyl)-4-phenylbutan-2-yl) acetamide of step is treated with an acetylating agent in an organic solvent.

The acetylating agent used is selected from acetic anhydride, acetyl chloride, acetyl bromide.

In a particular embodiment of the present invention the acetylating agent used is acetic anhydride. The use of acetic anhydride over acetyl chloride or acetyl bromide is preferred because of less hazardousness and cheaper than the other reagents. Inventors have observed the reactions in which acetyl chloride or acetyl bromide is used results in low purity due to formation of large number of byproducts.

The step c) reaction was observed preferably to be performed in an inert atmosphere with acetic anhydride in pyridine.

From various organic solvent like of pyridine, dichloromethane, chloroform, tetrahydrofuran, dioxane and toluene, pyridine is found to be more suitable for the acetylation. Inventors have found the extent of acetylation reaction is less in other solvents in comparison to pyridine. Pyridine not only work as solvent, it also accelerates the reaction rate as catalyst.

The step c) of the present invention is performed at 25-30° C. for 2-6 hrs, more particularly, for 4 hrs.

In presence of pyridine with acetic anhydride, inventors found that the acetylation occurs at a faster rate and reaction complies in about 4-5 hours at 25-30° C.

The isolated product of step c) of the present invention is highly pure intermediate [2-acetamido-2-(acetyloxy methyl)-4-phenylbutyl] acetate (II) having purity exceeding 98% (by HPLC).

In step d) of the present invention the isolated highly pure key intermediate i.e. [2-acetamido-2-(acetyloxy methyl)-4-phenylbutyl] acetate(II) is further converted to Fingolimod Hydrochloride of formula (I).

In another embodiment, the final product Fingolimod HCl obtained by the processes of the present application may be formulated as solid compositions for oral administration in the form of capsules, tablets, pills, powders or granules useful in the treatment or prevention of autoimmune related disorder including multiple sclerosis. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium. Pharmaceutically acceptable excipients used in the compositions comprising highly pure Fingolimod HCl obtained by the process of the present invention include, but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from highly pure Fingolimod HCl obtained by the process of the present invention may also comprise to include the pharmaceutically acceptable carrier used for the preparation of solid dispersion, wherever utilized in the desired dosage form preparation.

Certain specific aspects and embodiments of the present application will be explained in more detail with reference to the following example, which is provided by way of illustration only and should not be construed as limiting the scope of the invention in any manner.

### EXAMPLES

### Example 01: Preparation of Highly Pure Intermediate-[2-acetamido-2-(acetyloxy methyl)-4-phenylbutyl] acetate(II)

### Step a: Preparation of Diethyl 2-acetamido-2-phenethylmalonate (IV)

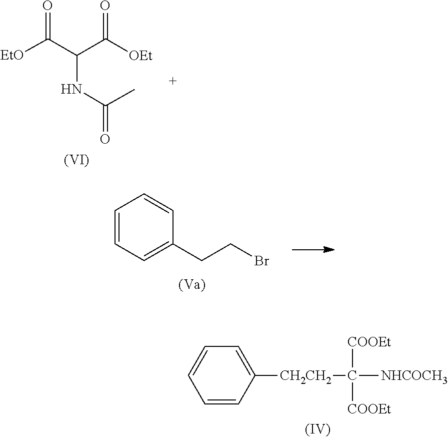

Under nitrogen, charge 850 ml dimethyl sulfoxide in a 2.0 litre round-bottom flask. Charge 100 gm diethylacetamidomalonate(VI) at 25-30° C. Stir the reaction mass for 10-15 minutes. Add 195 gm cesium carbonate and tetrabutylammonium bromide 7.5 gm. Stir the reaction mass for 60-90 minutes. Gradually, add 90 gm phenylethyl bromide(Va) within a time interval of 60-120 minutes at 25-35° C. After complete addition of phenylethylbromide, raise the temperature of the reaction mass to 80-85° C. Maintain under nitrogen for 6 hours at 80-85° C. After reaction completion, charge 2125 ml purified water in a 5.0 litre round bottom flask and cool to 0-10° C. Slowly, charge the reaction mass into pre-cooled water in 50-60 minutes at 0-10° C. Stir the reaction mass for 90-120 minutes at 0-10° C. Filter and wash the wet cake with 1 litre purified water. Suck dry and unload the wet cake. Further, vacuum dry the wet cake at 55-55° C. for 2-3 hours.

Charge the dry cake (115 gm) in 450 ml of isopropyl alcohol in a 2.0 liter round bottom flask. Slowly, heat the reaction mass to 55-65° C. and stir till a clear solution is observed. Gradually cool to 5-10° C. and maintain for 2-2.5 hours. Filter and wash with 75 ml chilled isopropyl alcohol. Suck dry and unload the wet cake. Dry under vacuum at 50-60° C. for 8-10 hours.

Dry weight—95 gm

Purity: 98.3% (by HPLC)

Heating weight loss (%): 0.201

### Step b: Preparation of N-(1-hydroxy-2-(hydroxymethyl)-4-phenylbutan-2-yl) Acetamide (III)

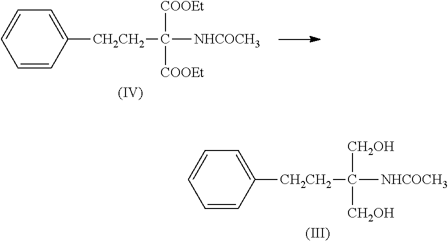

In 500 ml of methanol, charge 50 gm of diethyl 2-acetamido-2-phenethylmalonate (IV) prepared in the above step at 25-30° C. Stir the reaction mass and cool to 0-5° C. Slowly, charge the solution of sodium borohydride in water (58.9 gm in 100 ml purified water) in 30-40 minutes at 0-5° C. Raise the temperature of the reaction mass to 50-60° C. and maintain for 4-6 hours. Cool to 0-10° C. and adjust pH to 7.0 by aqueous hydrochloric acid solution. Raise the temperature of the reaction mass to 25-30° C. and distill out methanol under vacuum at 40-45° C. Charge 300 ml purified water to the residue and extract three times with 500 ml ethyl acetate each. Combine the organic layer and wash with 500 ml of 20% of sodium chloride solution. Dry the organic layer over sodium sulfate and distill out under vacuum at 40-45° C. Proceed with the residue to the next step.

Weight of the residue: 34.5 gm

### Purity: 94.56% (by HPLC)

### Step c: Preparation of Highly Pure Intermediate-[2-acetamido-2-(acetyloxy methyl)-4-phenylbutyl] Acetate(II)

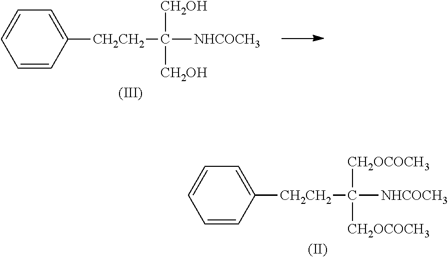

Under nitrogen atmosphere, charge the residue obtained in the step b) in 125 ml of pyridine. Stir the mass till clear solution is observed. Cool to 0-5° C. and slowly add 88 ml of acetic anhydride in 20-30 minutes. Raise the temperature of the reaction mass to 20-30° C. and stir for 5-6 hours. After completion of reaction, charge 625 ml of purified water and 625 ml of ethyl acetate into the reaction mass. Stir and separate layers. Wash the aqueous layer again with 625 ml of ethyl acetate. Stir and separate layers. Combine both the organic layers and wash with 375 ml of 30% ammonium chloride solution. Again wash the organic layer with 125 ml of purified water, stir and separate layers. Dry the organic layer over sodium sulfate and filter through hyflo bed. Distill out the ethyl acetate under vacuum with twice toluene striping of 200 ml each at 40-50°. Charge 250 ml toluene to the degas mass and heat to 60-70°. After a clear solution is observed, gradually cool to 20-30° C. and maintain for 2-3 hours. Filter and wash with 50 ml×2 toluene. Suck dry and then vacuum dry the wet cake for 8-10 hours at 50-60° C. Recrystallising the dried material using toluene to get pure material.

Dry weight: 25 gm

### HPLC Purity: 99.584%

### Example 02: Preparation of Highly Pure Intermediate-[2-acetamido-2-(acetyloxy methyl)-4-phenylbutyl] Acetate(II)

### Step a: Preparation of Diethyl 2-acetamido-2-phenethylmalonate (IV)

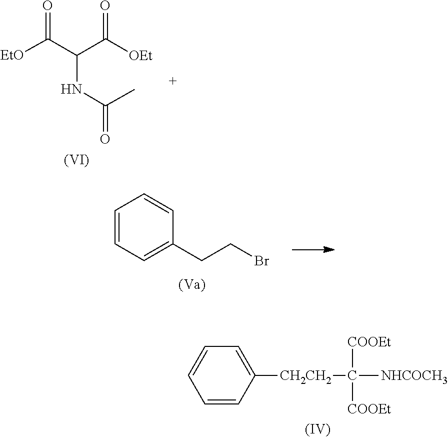

Under nitrogen, charge 2125 ml dimethyl sulfoxide in a 5.0 litre round-bottom flask. Charge 250 gm diethylacetamidomalonate at 25-30° C. Stir the reaction mass for 10-15 minutes. Add 487.1 gm cesium carbonate and tetrabutylammonium bromide 19 gm. Stir the reaction mass for 60-90 minutes. Gradually, add 224 gm phenylethyl bromide within a time interval of 60-120 minutes at 25-35° C. After complete addition of phenylethylbromide, raise the temperature of the reaction mass to 80-85° C. Maintain under nitrogen for 6 hours at 80-85° C. After reaction completion, charge 5300 ml purified water in a 10.0 litre round bottom flask and cool to 0-10° C. Slowly, charge the reaction mass into pre-cooled water in 50-60 minutes at 0-10° C. Stir the reaction mass for 90-120 minutes at 0-10° C. Filter and wash the wet cake with 2.5 litre purified water. Suck dry and unload the wet cake. Further, vacuum dry the wet cake at 55-55° C. for 6-8 hours.

Charge the dry cake (295 gm) in 1125 ml of isopropyl alcohol in a 2.0 liter round bottom flask. Slowly, heat the reaction mass to 55-65° C. and stir till a clear solution is observed. Gradually cool to 5-10° C. and maintain for 2-2.5 hours. Filter and wash with 190 ml chilled isopropyl alcohol. Suck dry and unload the wet cake. Dry under vacuum at 50-60° C. for 8-10 hours.

Dry weight-237 gm; HPLC purity:

### Step b: Preparation of N-(1-hydroxy-2-(hydroxymethyl)-4-phenylbutan-2-yl) Acetamide (III)

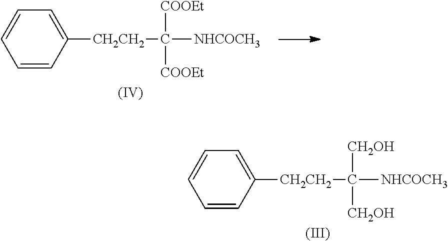

In 1000 ml of methanol, charge 100 gm of diethyl 2-acetamido-2-phenethylmalonate (IV) prepared in the above step at 25-30° C. Stir the reaction mass and cool to 0-5° C. Slowly, charge the solution of sodium borohydride in water (118 gm in 200 ml purified water) in 30-40 minutes at 0-5° C. Raise the temperature of the reaction mass to 40-60° C. and maintain for 3-4 hours. Cool the reaction mass to 0-10° C. and adjust pH to about 7.0 by aqueous hydrochloric acid solution. Raise the temperature of the reaction mass to 20-30° C. and distill out methanol under vacuum at 40-45° C. Charge 1600 ml purified water to the residue and extract three times with 1 litre ethyl acetate each. Combine the organic layer and wash with 500 ml of 20% of sodium chloride solution. Dry the organic layer over sodium sulfate and distill out under vacuum at 40-50° C. Proceed with the residue to the next step.

### Step c: Preparation of Highly Pure Intermediate-[2-acetamido-2-(acetyloxy methyl)-4-phenylbutyl] Acetate(II)

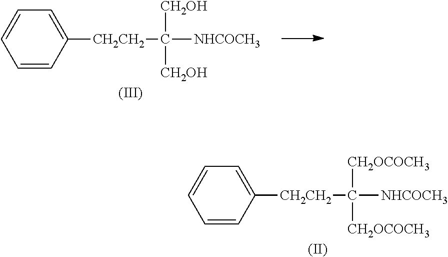

Under nitrogen atmosphere, charge the residue obtained in the step b) in 250 ml of pyridine. Stir the mass till clear solution is observed. Cool to 0-5° C. and slowly add 176 ml of acetic anhydride in 20-30 minutes. Raise the temperature of the reaction mass to 20-30° C. and stir for 5-6 hours. After completion of reaction, charge 1250 ml of purified water and 1250 ml of ethyl acetate into the reaction mass. Stir and separate layers. Wash the aqueous layer again with 250 ml of ethyl acetate. Stir and separate layers. Combine both the organic layers and wash with 750 ml of 30% ammonium chloride solution. Again wash the organic layer with 250 ml of purified water, stir and separate layers. Dry the organic layer over sodium sulfate and filter through hyflo bed. Distill out the ethyl acetate under vacuum with twice toluene striping of 200 ml each at 40-50°. Charge 500 ml toluene to the degas mass and heat to 60-70°. After a clear solution is observed, gradually cool to 20-30° C. and maintain for 2-3 hours. Filter and wash with 50 ml×2 toluene. Suck dry and then vacuum dry the wet cake for 8-10 hours at 50-60° C. Recrystallising the dried material using toluene to get pure material.

Dry weight: 40 gm. HPLC Purity: 99.58%

### Example 03: Preparation of Fingolimod Hydrochloride (I)

### Step a) Preparation of 2-acetamido-2-(4-octanoylphenethyl) propane-43-diyl Diacetate

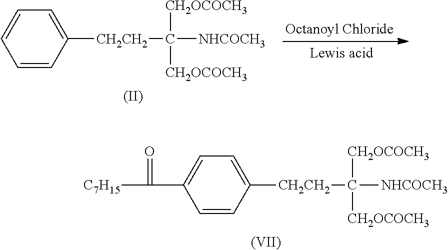

EDC (200 ml) was charged in a four necked RB flask and cooled up to −10° C. to −15° C., and under nitrogen atmosphere. Further Aluminium chloride (33.2 g) was added to the solution and stirred the reaction for 30 min. To this, octanoyl chloride (26.8 ml) was slowly added in an hour and the resulting reaction mixture stirred further for 1.5 hours, at −10° C. to −15° C. A solution of [2-acetamido-2-(acetyloxy methyl)-4-phenylbutyl] acetate(II) (10 g dissolved in 40.0 ml EDC) was slowly added to the reaction mixture over a period of around 3 hours, maintaining the temperature between −10° C. to −15° C. The reaction mixture was then allowed to come to room temperature and stirred overnight for 16 hours. After completion of reaction as confirmed by HPLC, the reaction mixture was slowly poured into chilled water (200 ml) and stirred for 15-20 min. The EDC layer was separated and the aqueous layer was extracted with EDC (2×200 ml). The organic layers were combined, washed with saturated sodium chloride solution (2×50 ml), dried over anhydrous sodium sulfate (10 g), filtered and concentrated under vacuum at temperature below 50° C. to get residue. The residue was purified by adding Hexane (200 ml) and stirred the solution for about 2 hours to provide white crystalline solid. The crystalline solid purified material was filtered and suck dried for to afford 13.0 g title compound. By this surprising process, no column purification is desired, which is clumsy and time incurring step.

### Yield: 93.52%

### Purity (by HPLC): 85.49%

Ortho isomer impurity: 6.280%

### Step b) Preparation of 2-acetamido-2-(4-octylphenethyl) propane-1, 3-diyl Diacetate

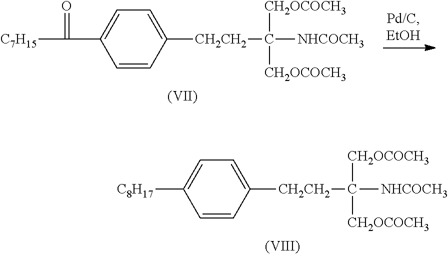

Charged 13 g of 2-acetamido-2-(4-octanoylphenethyl) propane-1, 3-diyl diacetate and ethanol (152.10 ml) in a 2 L steel hydrogenation vessel (autoclave), followed by addition of 10% Pd—C(2.73 g). The reaction mixture was hydrogenated (4 kg/cm² 112 pressure) at RT for 2-3 h. The progress of the reaction was confirmed by HPLC. After completion of the reaction, the reaction mixture was filtered through hyflo bed & washed with Ethanol (20 ml). The filtrate was concentrated under reduced pressure below 50° C. to give 14.5 g of residue. Hexane (98 ml) was added to and stirred for 2-3 hours at RT. The separated white solid material was filtered to give 8.6 g crude 2-acetamido-2-(4-octylphenethyl) propane-1, 3-diyl diacetate compound (HPLC purity=97.25%). The obtained crude title compound was taken in methanol (103 ml) and stirred for 30 min at RT to get the clear solution. The solution was then cooled to 0-5° C. under stirring maintaining for two hours. The solid obtained was washed with chilled methanol, filtered and dried at 45° C. under vacuum for 2-3 hours, to obtain 6.3 g of title compound.

Yield: 50%

### Purity (by HPLC): 99.01%

### Step c) Preparation of Fingolimod Free Base

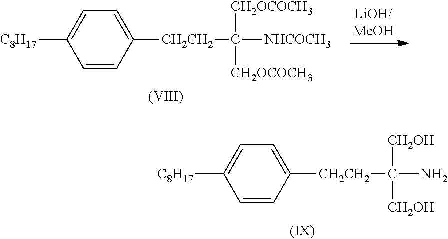

6 gm of 2-acetamido-2-(4-octylphenethyl) propane-1, 3-diyl diacetate was charged in a three necked RB flask, further added methanol (78 ml) and stirred at room temp till solution becomes clear. To this slowly added LiOH solution (12.2 g dissolved in 78 ml of DM Water) and then the reaction mixture stirred at reflux for 2-3 hours. The completion of reaction was monitored by HPLC. The reaction mixture was concentrated under reduced pressure below 45° C. to give residue which was taken in DM water (42 ml) and extracted twice with ethyl acetate (215 ml and 107.5 ml). The ethyl acetate layers were combined, washed with saturated brine (30 ml), dried over sodium sulfate (21 g) and concentrated under vacuum below 45° C. Ethyl acetate (12 ml) was added to residue and cooled the solution to 0-5° C., maintained the temperature for 2 hours and then filtered to give 3.6 g. Fingolimod free base, which was further dried under vacuum at 45° C. for 6-7 hours.

### Yield: 84.6%

### Purity (by HPLC): 99.84%

### Step d) Preparation of Fingolimod Hydrochloride

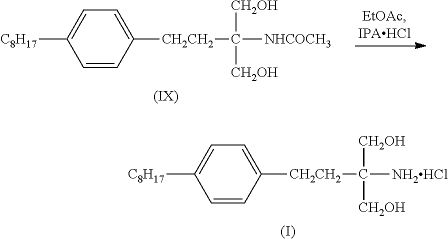

In a three necked round bottom flask Fingolimod free base (3.5 g) was charged to ethyl acetate (89 ml) and reaction stirred at room temperature for 15-20 min. The reaction mixture was slowly heated up to reflux (˜70° C.) to get a clear solution. A mixture of 10% IPA-HCl (23 ml) Aqueous IPA-HCl solution was added to the reaction mixture at 50° C. over 10-15 min till pH 1-2 and resulting solution further stirred for 30 minutes. The reaction temperature was then cooled to 0-5° C. and maintained for 2-3 hours. The separated solid was filtered & washed with cold ethyl acetate to get the title compound. The material obtained was dried at 45° C. under full vacuum for 6-7 hours to get 3.4 g of Fingolimod hydrochloride.

### Yield: 86.03%

### Purity (by HPLC): 99.96%

While the forgoing pages provide a detailed description of the preferred embodiments of the invention, it is being understood that the summary, description and examples are illustrative only of the core of the invention and non-limiting in nature. Furthermore, as many changes can be made to the invention without departing from the scope of the invention, it is intended that all material contained herein shall be interpreted as illustrative of the invention and not in a limiting sense.

We claim:

1. A process for the preparation of Fingolimod Hydrochloride of formula (I): 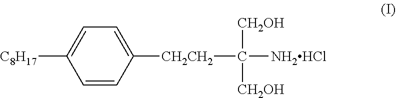
comprising of the following steps:
a) reacting diethylacetamidomalonate(VI) with phenylethylhalide(V) in presence of a phase transfer catalyst, base and polar aprotic solvent at temperature ranging between 80-100° C. for a time ranging between 4-8 hours to give diethyl 2-acetamido-2-phenethylmalonate (IV). 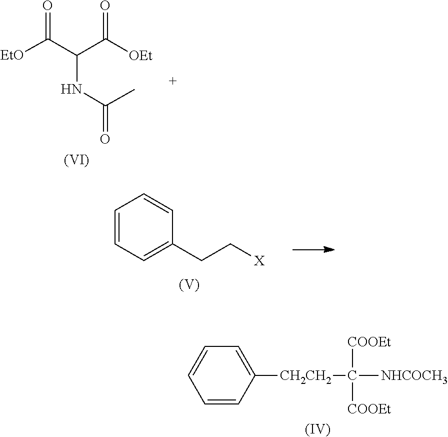
b) reacting diethyl 2-acetamido-2-phenethylmalonate(IV) with aqueous NaBH₄ at temperature ranging between 30-70° C. to give N-(1-hydroxy-2-(hydroxymethyl)-4-phenylbutan-2-yl) acetamide (III). 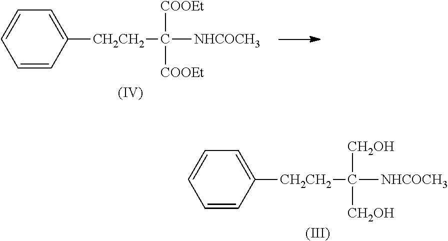
c) acetylation of N-(1-hydroxy-2-(hydroxymethyl)-4-phenylbutan-2-yl) acetamide(III) with an acetylating agent in presence of an organic solvent for time duration ranging between 2-6 hrs to give [2-acetamido-2-(acetyloxy methyl)-4-phenylbutyl] acetate (II). 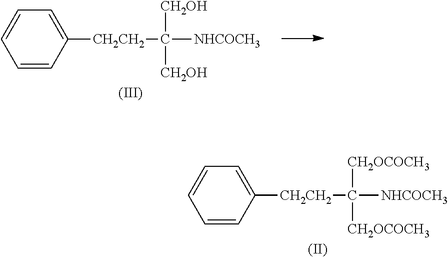
d) the converting [2-acetamido-2-(acetyloxy methyl)-4-phenylbutyl] acetate(II) obtained in step c) to get Fingolimod Hydrochloride of formula (I).

2. A process for the preparation of Fingolimod Hydrochloride of formula (I) according to claim 1, wherein phase transfer catalyst used in step a) is selected from tetrabutylammonium bromide, benzyltrimethylammonium bromide, hexyltrimethylammonium bromide.

3. A process for the preparation of Fingolimod Hydrochloride of formula (I) according to claim 1, wherein phenylethylhalide used in step a) is selected from phenylethylbromide, phenylethyliodide, phenylethylchloride.

4. A process for the preparation of Fingolimod Hydrochloride of formula (I) according to claim 1, wherein base used in step a) is selected from cesium carbonate, lithium carbonate.

5. A process for the preparation of Fingolimod Hydrochloride of formula (I) according to claim 1, wherein polar aprotic solvent used in step a) is selected from dimethyl sulfoxide, dimethylformamide, ethyl acetate.

6. A process for the preparation of Fingolimod Hydrochloride of formula (I) according to claim 1, wherein step b) is performed in aqueous alcoholic medium comprising a ratio of alcohol (C₁-C₃):water mixture is ranging between 1(diethyl 2-acetamido-2-phenethyl malonate(IV)):10-15(alcohol) (w/v) and 1(diethyl 2-acetamido-2-phenethylmalonate(IV)):2-3(water) (w/v).

7. A process for the preparation of Fingolimod Hydrochloride of formula (I) according to claim 1, wherein acetylating agent used in step c) is selected from acetic anhydride, acetyl chloride, acetyl bromide.

8. A process for the preparation of Fingolimod Hydrochloride of formula (I) according to claim 1, wherein organic solvent used in step c) is selected from pyridine, dichloromethane, chloroform, tetrahydrofuran, dioxane, toluene.

9. Highly pure intermediate of compound of formula II 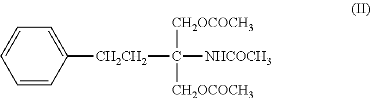
having purity exceeding 98% (by HPLC).
